(19) United States
(12) Patent Application Publication
(10) Pub. No.: US 2024/0316055 A1
(43) Pub. Date: Sep. 26, 2024

(54) METHODS FOR TREATING LEIGH SYNDROME

(71) Applicant: DBCC Research, LLC, San Mateo, CA (US)

(72) Inventors: Jeannie CHOI, San Mateo, CA (US); Giacomo COSLOVICH, San Mateo, CA (US); Ethan Oren PERLSTEIN, Oakland, CA (US)

(21) Appl. No.: 18/607,228

(22) Filed: Mar. 15, 2024

Related U.S. Application Data
(60) Provisional application No. 63/490,747, filed on Mar. 16, 2023.

(51) Int. Cl. A61K 31/519; A61K 31/437; A61K 31/4418; A61K 31/496; A61K 31/51

(52) U.S. Cl. CPC A61K 31/519; A61K 31/437; A61K 31/4418; A61K 31/496; A61K 31/51

(57) ABSTRACT

The present disclosure relates generally to methods for treating diseases or conditions mediated. at least in part, by ECHS1 enzyme. such as Leigh syndrome.

### CROSS-REFERENCE TO RELATED APPLICATIONS

This application claims the benefit under 35 U.S.C. § 119(e) of U.S. Provisional Application Ser. No. 63/490,747 filed Mar. 16, 2023, the contents of which are hereby incorporated by reference in its entirety.

### FIELD

Provided herein are compounds and pharmaceutical compositions for modulating ECHS1 enzyme activity and for treating Leigh syndrome.

### BACKGROUND

Leigh syndrome (LS) is an inherited neurological disorder caused by mitochondrial dysfunction. LS affects the central nervous system and is characterized by progressive loss of mental and motor capabilities. In most cases, LS is early-onset and is typically diagnosed in infancy (also known as classic LS or infantile necrotizing encephalopathy), leading to death within several years. In rare cases, symptoms do not appear until adolescence or early adulthood.

LS has been associated with a mutation in the ECHS1 gene, which encodes a short-chain enoyl-CoA hydratase that is involved in the metabolism of fatty acids and branched-chain amino acids in mitochondria. Mutations in the ECHS1 gene disrupts the activity of the mitochondrial respiratory complex of cells in the brain stem and basal ganglia, leading to cell death and impaired motor functions. It is estimated that approximately 1 in 40,000 newborns are affected by LS. Currently, there is no known cure for LS.

### SUMMARY

It has now been found that certain classes of compounds will be useful in treating diseases associated with ECHS1 mutation, such as Leigh syndrome, due to their ability to modulate the mutant enzyme activity and restore fatty acid metabolism. Thus, the present disclosure provides compounds and methods for modulating the activity of the ECHS1 enzyme. Abnormal ECHS1 enzyme function has been associated with disruptions in mitochondrial function, leading to cell death in the central nervous system. Disclosed herein are compounds and methods useful for treating neurological diseases or conditions associated with ECHS1 enzyme, such as Leigh syndrome.

The present disclosure provides methods for treating a disease or condition mediated, at least in part, by ECHS1 enzyme in a patient in need thereof, the method comprising administering a therapeutically effective amount a compound selected from a p38 MAPK inhibitor, casein kinase inhibitor, TGF-β receptor inhibitor, RAF inhibitor, serine/threonine kinase inhibitor, TIE tyrosine kinase inhibitor, monocarboxylate transporter inhibitor, tyrosine phosphatase inhibitor, IAP inhibitor, CDK inhibitor, cytokine production inhibitor, Abl kinase inhibitor, bone morphogenetic protein inhibitor, AMPK inhibitor, MTH1 inhibitor, DYRK inhibitor, deubiquitinase inhibitor, ICAM1 expression inhibitor, calcium channel blocker, glucosylglucose, trace amine-associated receptor antagonist, a G protein-coupled receptor agonist, or a pharmaceutically acceptable salt thereof, or a combination thereof.

In one embodiment, the compound is ralimetinib, talmapimod, SB-239063, neflamapimod, SB-202190, TA-01, SB-242235, AL-8697, losmapimod, RepSox, tovorafenib, FRAX486, pexmetinib, adezmapimod, AZD3965, GW-1100, PPT, RWJ-67657, SX-011, felodipine, D-(+)-maltose, SKF-86002, TAK-715, bis(maltolato)oxovanadium(IV), EO-1428, SM-164, BMS-265246, semapimod, LY2109761, GDC-0879, PPY-A, K02288, WZ4003, SCH-51344, ID-8, AZD5582, CUDC-427, spautin-1, A205804, or SB590885, or a pharmaceutically acceptable salt thereof, or a combination thereof. In one embodiment, the compound is a p38 MAPK inhibitor. In one embodiment, the p38 MAPK inhibitor is selected from losmapimod, talmapimod, ralimetinib, pexmetinib, talmapimod, or neflamapimod. In one embodiment, the compound is administered in a pharmaceutical composition comprising a therapeutically effective amount of the compound and a pharmaceutically acceptable excipient. In one embodiment, the disease or condition is a neurodegenerative disease or condition. In one embodiment, the neurodegenerative disease or condition is Leigh syndrome. In one embodiment, the patient is further administered a therapeutically effective amount of an additional therapeutic agent. In one embodiment, the additional therapeutic agent is thiamine. In one embodiment, the patient is less than 3 years old.

Also provided herein are methods for treating Leigh Syndrome comprising administering to a patient in need thereof, a therapeutically effective amount of a p38 MAPK inhibitor, or a pharmaceutically acceptable salt thereof.

In one embodiment, the compound is administered in a pharmaceutical composition comprising a therapeutically effective amount of the compound and a pharmaceutically acceptable excipient. In one embodiment, the p38 MAPK inhibitor is selected from losmapimod, talmapimod, ralimetinib, pexmetinib, talmapimod, or neflamapimod. In one embodiment, the patient is further administered a therapeutically effective amount of an additional therapeutic agent. In one embodiment, the additional therapeutic agent is thiamine. In one embodiment, the patient is less than 3 years old.

The present disclosure provides methods for treating Leigh syndrome comprising administering to a patient in need thereof a therapeutically effective amount of a compound which is selected from:

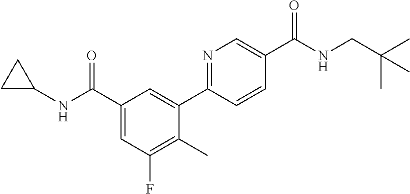 [SMILES: Cc1c(F)cc(C(=O)NC2CC2)cc1-c1ccc(C(=O)NCC(C)(C)C)cn1]

or a pharmaceutically acceptable salt thereof;

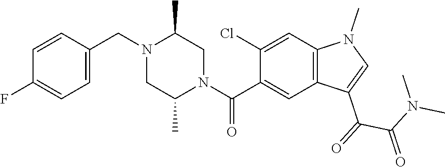 [SMILES: C[C@@H]1CN(Cc2ccc(F)cc2)[C@@H](C)CN1C(=O)c1cc2c(C(=O)C(=O)N(C)C)cn(C)c2cc1Cl]

or a pharmaceutically acceptable salt thereof;

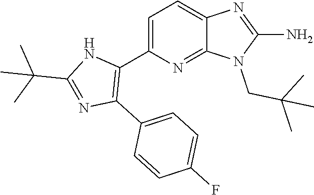 [SMILES: CC(C)(C)Cn1c(N)nc2ccc(-c3[nH]c(C(C)(C)C)nc3-c3ccc(F)cc3)nc21]

or a pharmaceutically acceptable salt thereof;

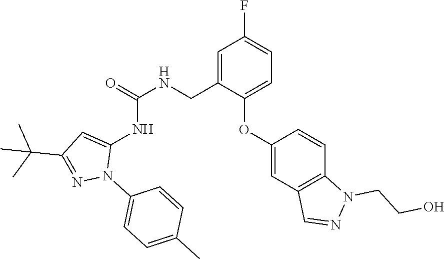 [SMILES: Cc1ccc(-n2nc(C(C)(C)C)cc2NC(=O)NCc2cc(F)ccc2Oc2ccc3c(cnn3CCO)c2)cc1]

or a pharmaceutically acceptable salt thereof; and

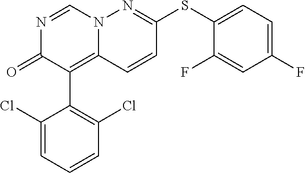 [SMILES: O=c1ncn2nc(Sc3ccc(F)cc3F)ccc2c1-c1c(Cl)cccc1Cl]

or a pharmaceutically acceptable salt thereof.

In one embodiment, the patient is further administered a therapeutically effective amount of an additional therapeutic agent. In one embodiment, the additional therapeutic agent is thiamine. In one embodiment, the patient is less than 3 years old.

### BRIEF DESCRIPTION OF THE DRAWINGS

FIG. 1 shows the DAPI-stained raw nuclei image of cells treated with compound SB-239063 from the galactose potency screen (Example 3), shown alongside the positive and negative control experiments.

FIG. 2 shows the concentration-dependent curves of LY2228820, talmapimod, SB-239063, and VX-745 from the galactose potency screen (Example 3), showing a non-linear response.

FIG. 3 shows the DAPI-stained raw nuclei image of cells treated with compound WZ4003 from the propionate potency screen (Example 3) shown alongside the positive and negative control experiments.

FIG. 4 shows the concentration-dependent curves of SM-164, BMS-265246, pexmetinib, WZ4003, and ID-8 from the propionate potency screen (Example 3), showing a non-linear response.

FIG. 5 shows the larval size measured as described in Example 4, at various concentrations of RepSox, TA-01, felodipine, pexmetinib, ID-8, talmapimod, neflamapimod, ralimetinib, losmapimod, and semapimod.

FIG. 6 shows the effect of losmapimod, talmapimod, ralimetinib, and neflamamipod on ECHS1 RNAi buoyancy phenotype as described in Example 4.

FIG. 7 shows the effect of losmapimod, talmapimod, ralimetinib, and neflamamipod on ECHS1 RNAi small size phenotype as described in Example 4.

### DETAILED DESCRIPTION

### Definitions

The following description sets forth exemplary embodiments of the present technology. It should be recognized, however, that such description is not intended as a limitation on the scope of the present disclosure but is instead provided as a description of exemplary embodiments.

As used in the present specification, the following words, phrases and symbols are generally intended to have the meanings as set forth below, except to the extent that the context in which they are used indicates otherwise.

Reference to “about” a value or parameter herein includes (and describes) embodiments that are directed to that value or parameter per se. In certain embodiments, the term “about” includes the indicated amount ±10%. In other embodiments, the term “about” includes the indicated amount ±5%. In certain other embodiments, the term “about” includes the indicated amount ±1%. In certain other embodiments, the term “about” includes the indicated amount ±0.05%. Also, to the term “about X” includes description of “X.”

Also, the singular forms “a” and “the” include plural references unless the context clearly dictates otherwise. Thus, e.g., reference to “the compound” includes a plurality of such compounds and reference to “the assay” includes reference to one or more assays and equivalents thereof known to those skilled in the art.

Provided are also pharmaceutically acceptable salts, stereoisomers, mixture of stereoisomers, hydrates, solvates, solid forms, and tautomeric forms of the compounds described herein.

In many cases, the compounds of this disclosure are capable of forming acid and/or base salts by virtue of the presence of amino and/or carboxyl groups or groups similar thereto.

“Pharmaceutically acceptable” or “physiologically acceptable” refer to compounds, salts, compositions, dosage forms and other materials which are useful in preparing a pharmaceutical composition that is suitable for veterinary or human pharmaceutical use.

The term “pharmaceutically acceptable salt” of a given compound refers to salts that retain the biological effectiveness and properties of the given compound, and which are not biologically or otherwise undesirable. “Pharmaceutically acceptable salts” or “physiologically acceptable salts” include, for example, salts with inorganic acids and salts with an organic acid. In addition, if the compounds described herein are obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare nontoxic pharmaceutically acceptable addition salts. Pharmaceutically acceptable acid addition salts may be prepared from inorganic and organic acids. Salts derived from inorganic acids include hydrochloric acid, hydrobromic acid, sulfuric acid, nitric acid, phosphoric acid, and the like. Salts derived from organic acids include acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like. Likewise, pharmaceutically acceptable base addition salts can be prepared from inorganic and organic bases. Salts derived from inorganic bases include, by way of example only, sodium, potassium, lithium, ammonium, calcium, and magnesium salts. Salts derived from organic bases include, but are not limited to, salts of primary, secondary, and tertiary amines. Specific examples of suitable amines include, by way of example only, isopropylamine, trimethyl amine, diethyl amine, tri(iso-propyl) amine, tri(n-propyl) amine, ethanolamine, 2-dimethylaminoethanol, piperazine, piperidine, morpholine, N-ethylpiperidine, and the like.

The term “solvate” refers to a complex formed by a combination of solvent molecules with molecules or ions of the solute. The solvent can be an organic compound, an inorganic compound, or a mixture of both. As used herein, the term “solvate” includes a “hydrate” (i.e., a complex formed by combination of water molecules with molecules or ions of the solute), hemi-hydrate, channel hydrate, etc. Some examples of solvents include, but are not limited to, methanol, N,N-dimethylformamide, tetrahydrofuran, dimethylsulfoxide, and water. In general, the solvated forms are equivalent to unsolvated forms and are encompassed within the scope of the present disclosure.

The term “solid form” refers to a type of solid-state material that includes amorphous as well as crystalline forms. The term “crystalline form” refers to polymorphs as well as solvates, hydrates, etc. The term “polymorph” refers to a particular crystal structure having particular physical properties such as X-ray diffraction, melting point, and the like.

Some of the compounds exist as tautomers. Tautomers are in equilibrium with one another. For example, amide containing compounds may exist in equilibrium with imidic acid tautomers. Regardless of which tautomer is shown, and regardless of the nature of the equilibrium among tautomers, the compounds are understood by one of ordinary skill in the art to comprise both amide and imidic acid tautomers. Thus, the amide containing compounds are understood to include their imidic acid tautomers. Likewise, the imidic acid containing compounds are understood to include their amide tautomers.

Any formula or structure given herein, is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the disclosure include isotopes of hydrogen, carbon, nitrogen, oxygen, phosphorous, fluorine and chlorine, such as, but not limited to ²H (deuterium, D), ³H (tritium), ¹¹C, ¹³C, ¹⁴C, ¹⁵N, ¹⁸F, ³¹P, ³²P, ³⁵S, ³⁶Cl and ¹²⁵¹. Various isotopically labeled compounds of the present disclosure include, for example, those into which radioactive isotopes such as ³H, ¹³C and ¹⁴C, are incorporated. Such isotopically labelled compounds may be useful in metabolic studies, reaction kinetic studies, detection, or imaging techniques, such as positron emission tomography (PET) or single-photon emission computed tomography (SPECT) including drug or substrate tissue distribution assays or in radioactive treatment of patients.

The disclosure also includes “deuterated analogs” of compounds described herein in which from 1 to n hydrogens attached to a carbon atom is/are replaced by deuterium, in which n is the number of hydrogens in the molecule. Such compounds exhibit increased resistance to metabolism and are thus useful for increasing the half-life of any compound described herein when administered to a mammal, particularly a human. See, for example, Foster, “Deuterium Isotope Effects in Studies of Drug Metabolism,” Trends Pharmacol. Sci. 5(12):524-527 (1984). Such compounds are synthesized by means well known in the art, for example by employing starting materials in which one or more hydrogens have been replaced by deuterium.

Deuterium labelled or substituted therapeutic compounds of the disclosure may have improved DMPK (drug metabolism and pharmacokinetics) properties, relating to distribution, metabolism, and excretion (ADME). Substitution with heavier isotopes such as deuterium may afford certain therapeutic advantages resulting from greater metabolic stability, for example increased in vivo half-life, reduced dosage requirements and/or an improvement in therapeutic index. An ¹⁸F labeled compound may be useful for PET or SPECT studies. Isotopically labeled compounds of this disclosure can generally be prepared by carrying out syntheses known in the art and substituting a readily available isotopically labeled reagent for a non-isotopically labeled reagent.

The concentration of such a heavier isotope, specifically deuterium, may be defined by an isotopic enrichment factor. In the compounds of this disclosure, any atom not specifically designated as a particular isotope is meant to represent any stable isotope of that atom. Unless otherwise stated, when a position is designated specifically as “H” or “hydrogen”, the position is understood to have hydrogen at its natural abundance isotopic composition. Accordingly, in the compounds of this disclosure any atom specifically designated as a deuterium (D) is meant to represent deuterium.

As used herein, “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions.

As used herein, “modulation” or “modulating” means changing, or regulating, and may include inhibiting, enhancing, or supplementing the activity of a particular enzyme. Compounds may modulate the activity of an enzyme by increasing or decreasing its activity, inhibiting the particular enzyme altogether, or otherwise supplementing the normal activity of an enzyme by correcting a deficiency caused by abnormal activity of said enzyme. The compounds described herein may, in some embodiments, be administered to a subject who is suffering from a condition mediated by an enzymatic deficiency.

“Treatment” or “treating” is an approach for obtaining beneficial or desired results including clinical results. Beneficial or desired clinical results may include one or more of the following: a) inhibiting the disease or condition (e.g., decreasing one or more symptoms resulting from the disease or condition, and/or diminishing the extent of the disease or condition); b) slowing or arresting the development of one or more clinical symptoms associated with the disease or condition (e.g., stabilizing the disease or condition, preventing or delaying the worsening or progression of the disease or condition, and/or preventing or delaying the spread (e.g., metastasis) of the disease or condition); and/or c) relieving the disease, that is, causing the regression of clinical symptoms (e.g., ameliorating the disease state, providing partial or total remission of the disease or condition, enhancing effect of another medication, delaying the progression of the disease, increasing the quality of life, and/or prolonging survival).

“Prevention” or “preventing” means any treatment of a disease or condition that causes the clinical symptoms of the disease or condition not to develop. Compounds may, in some embodiments, be administered to a subject (including a human) who is at risk or has a family history of the disease or condition.

“Subject” or “patient” refers to an animal, such as a mammal (including a human), that has been or will be the object of treatment, observation, or experiment. The methods described herein may be useful in human therapy and/or veterinary applications. In some embodiments, the subject or patient is a mammal. In some embodiments, the subject or patient is a human.

The term “therapeutically effective amount” or “effective amount” of a compound described herein means an amount sufficient to effect treatment when administered to a subject, to provide a therapeutic benefit such as amelioration of symptoms or slowing of disease progression. For example, a therapeutically effective amount may be an amount sufficient to decrease a symptom of a condition or disorder described herein, including but not limited to Leigh syndrome. The therapeutically effective amount may vary depending on the subject, and disease or condition being treated, the weight and age of the subject, the severity of the disease or condition, and the manner of administering, which can readily be determined by one or ordinary skill in the art.

The methods described herein may be applied to cell populations in vivo or ex vivo. “In vivo” means within a living individual, as within an animal or human. In this context, the methods described herein may be used therapeutically in an individual. “Ex vivo” means outside of a living individual. Examples of ex vivo cell populations include in vitro cell cultures and biological samples including fluid or tissue samples obtained from individuals. Such samples may be obtained by methods well known in the art. Exemplary biological fluid samples include blood, cerebrospinal fluid, urine, and saliva. In this context, the compounds and compositions described herein may be used for a variety of purposes, including therapeutic and experimental purposes. For example, the compounds and compositions described herein may be used ex vivo to determine the optimal schedule and/or dosing of administration of a compound of the present disclosure for a given indication, cell type, individual, and other parameters. Information gleaned from such use may be used for experimental purposes or in the clinic to set protocols for in vivo treatment. Other ex vivo uses for which the compounds and compositions described herein may be suited are described below or will become apparent to those skilled in the art. The selected compounds may be further characterized to examine the safety or tolerance dosage in human or non-human subjects. Such properties may be examined using commonly known methods to those skilled in the art.

### Treatment Methods and Uses

Leigh syndrome (LS) is a pediatric mitochondrial disorder that affects the central nervous system. Affected individuals suffer from impaired mitochondrial function in neuronal cells, leading to cell death and subsequently impaired motor and mental functions.

Certain types of LS have been associated with mutations in the gene that encodes short-chain enoyl-CoA hydratase (ECHS1), an enzyme that is involved in mitochondrial metabolism (see, for example, Peters et al, Brain, 2014, 137 (Pt 11), 2903-2908 and Uchino et al, Human Genome Variation 6 (19), 2019). ECHS1 is active in several metabolic pathways in the mitochondria. In particular, ECHS1 catalyzes the second step in the beta-oxidation pathway of fatty acid metabolism, breaking down fatty acids into acetyl-CoA. It has been postulated that deficient ECHS1 activity not only leads to chronic energy deficiency in cells, but also a buildup of metabolites such as methacrylyl-CoA and acryloyl-CoA which are highly reactive and toxic intermediates.

It is hypothesized that certain compounds may be capable of restoring ECHSI enzyme activity. These compounds rescue or otherwise supplement mutant ECHS1 enzyme and enable the metabolism of fatty acids. A cell viability assay in a fatty acid-enriched medium may be used to screen for compounds capable of modulating ECHS1.

Provided herein are methods for modulating ECHS1 enzyme activity in a patient in need thereof, comprising administering a therapeutically effective amount of a compound as described herein, or a combination of the compounds described herein, or a composition as described herein.

Also provided herein are methods for treating a disease or condition mediated, at least in part, by ECHS1 enzyme in a patient in need thereof, comprising administering a therapeutically effective amount of a compound as described herein, or a combination of the compounds described herein, or a composition as described herein.

In some embodiments, the disease or condition mediated, at least in part, by ECHS1 enzyme, is a neurodegenerative disease or condition. In some embodiments, the neurodegenerative disease or condition is Leigh syndrome.

Provided herein are methods for treating Leigh syndrome, comprising administering a therapeutically effective amount of a compound as described herein, or a combination of compounds described herein, or a composition as described herein.

In any of the embodiments described herein, a patient is administered one or more of the compounds described herein. The one or more compounds can be administered simultaneously or sequentially.

In any of the embodiments described herein, a patient is administered a pharmaceutical composition that comprises one or more of the compounds described herein and a pharmaceutically acceptable excipient.

In any of the embodiments described herein, the patient is further administered a therapeutically effective amount of another therapeutic agent. In some embodiments, the another therapeutic agent is thiamine.

The another therapeutic agent may be administered simultaneously or sequentially with a compound, or compounds, described herein or a composition described herein.

In some embodiments the compound or pharmaceutical composition described herein is administered to a patient who is less than 3 years old.

### Compounds, Pharmaceutical Compositions, and Modes of Administration

Provided herein are compounds useful for modulating ECHSI enzyme activity.

Also provided herein are compounds useful for treating a disease or condition mediated, at least in part, by ECHS1 enzyme. Also provided herein are compounds useful for treating a neurological disease or condition. Also provided herein are compounds useful for treating Leigh syndrome.

In some embodiments, the compound is selected from a p38 MAPK inhibitor, casein kinase inhibitor, TGF-β receptor inhibitor, RAF inhibitor, serine/threonine kinase inhibitor, TIE tyrosine kinase inhibitor, monocarboxylate transporter inhibitor, tyrosine phosphatase inhibitor, IAP inhibitor, CDK inhibitor, cytokine production inhibitor, Abl kinase inhibitor, bone morphogenetic protein inhibitor, AMPK inhibitor, MTH1 inhibitor, DYRK inhibitor, deubiquitinase inhibitor, ICAM1 expression inhibitor, calcium channel blocker, glucosylglucose, trace amine-associated receptor antagonist, and a G protein-coupled receptor agonist.

In some embodiments, the compound is a p38 mitogen-activated protein kinase (p38 MAPK) inhibitor. A p38 MAPK inhibitor may prevent the production of proinflammatory cytokines by inhibiting the p38 MAPK-mediated signaling cascade. In some embodiments, the p38 MAPK inhibitor is selected from: as ralimetinib (also known as LY2228820), talmapimod, SB-239063, neflamapimod (also known as VX-745), SB-202190, TA-01, SB-242235, AL-8697, losmapimod, pexmetinib (also known as ARRY-614), adezmapimod (also known as SB-203580), RWJ-67657, SX-011, SKF-86002, TAK-715, EO-1428, or semapimod.

In some embodiments, the compound is a casein kinase inhibitor. Casein kinase inhibitors inhibit the activity of casein kinase 1 (CK1) and have been implicated in the treatment of various types of diseases including inflammatory, neurological, psychiatric, neurodegenerative, and/or ophthalmic diseases. In some embodiments, the casein kinase inhibitor is TA-01.

In some embodiments, the compound is a transforming growth factor beta (TGF-β) receptor inhibitor. TGF-β is a multifunctional cytokine that regulates stem cell and T-cell differentiation. TGF-β receptor inhibitors have been explored in therapies for various cancers, auto-immune diseases, and infectious diseases. In some embodiments, the TGF beta receptor inhibitor is RepSox or LY2109761.

In some embodiments, the compound is a RAF inhibitor. RAF inhibitors interfere with signal transduction pathways in cancer cells and have been evaluated widely as anticancer drugs. In some embodiments, the RAF inhibitor is selected from: tovorafenib (also known as TAK-580), GDC-0879, and SB-590885.

In some embodiments, the compound is a serine/threonine kinase inhibitor. Serine/threonine inhibitors, in particular p21-activated kinase inhibitors, have been implicated in various cancers and infectious diseases. In some embodiments, the serine/threonine kinase inhibitor is FRAX-486.

In some embodiments, the compound is a TIE tyrosine kinase inhibitor. TIE tyrosine kinase is expressed in vascular endothelial cells and tumor cells, and TIE tyrosine kinase inhibitors have been evaluated in antitumor therapies. In some embodiments, the TIE tyrosine kinase inhibitor is pexmetinib (also known as ARRY-614).

In some embodiments, the compound is a monocarboxylate transporter inhibitor. Monocarboxylate transporter 1 (MCT1) regulates cell metabolism and is a therapeutic target for cancer treatment. In some embodiments, the monocarboxylate transporter inhibitor is AZD3965.

In some embodiments, the compound is a tyrosine phosphatase inhibitor. Protein tyrosine phosphatases (PTP) have been implicated in various diseases such as diabetes mellitus, neural diseases such as Alzheimer's and Parkinson's disease, and cancer. In some embodiments, the tyrosine phosphate inhibitor is bis(maltolato)oxovanadium(IV).

In some embodiments, the compound is an IAP inhibitor. Inhibitors of apoptosis protein (IAP) are a family of antiapoptotic proteins which block cell death by controlling the caspase activation pathway. IAP inhibitors have been shown to lead to tumor regression in some studies. In some embodiments, the IAP inhibitor is SM-164, AZD5582, or CUDC-427.

In some embodiments, the compound is a CDK inhibitor. Cyclin-dependent kinase (CDK) inhibitors have been shown to prevent proliferation of cancer cells and have been evaluated in the treatment of estrogen receptor positive/HER2 negative breast cancer. In some embodiments, the CDK inhibitor is BMS-265246.

In some embodiments, the compound is a cytokine production inhibitor. Cytokine production inhibitors block abnormal production of proinflammatory cytokines and have been explored in treatments for various immunoinflammatory diseases. In some embodiments, the cytokine production inhibitor is semapimod.

In some embodiments, the compound is an Abl kinase inhibitor. Abelson (Abl) kinase has been implicated in the pathogenesis of chronic myelogenous leukemia in patients with a chromosomal abnormality, known as Philadelphia chromosome. In some embodiments, the Abl kinase inhibitor is PPY-A.

In some embodiments, the compound is a bone morphogenetic protein inhibitor. Bone morphogenetic protein (BMP) inhibitors have been studied for their potential use in the treatment of muscoskeletal diseases. In some embodiments, the bone morphogenic protein inhibitor is K02288.

In some embodiments, the compound is an AMPK inhibitor. 5′ adenosine monophosphate-activated protein kinase (AMPK) plays a key role in cellular energy regulation and is a therapeutic target for various conditions such as diabetes, inflammatory disease, and cancer. In some embodiments, the AMPK inhibitor is WZ4003.

In some embodiments, the compound is a MTH1 inhibitor. MutT homolog 1 (MTH1) is a member of the nudix phosphohydrolase family of enzymes that plays a role in DNA repair by hydrolyzing oxidized purines. MTH1 inhibitors have been studied for cancer therapies. In some embodiments, the MTH1 inhibitor is SCH-51344.

In some embodiments, the compound is a DYRK inhibitor. Dual specificity tyrosine-phosphorylation-regulated kinase (DYRK) is thought to play a role in signaling pathways regulating cell proliferation. In some embodiments, the DYRK inhibitor is ID-8.

In some embodiments, the compound is a deubiquitinase inhibitor. Deubiquitinases are proteases that cleave ubiquitin from proteins and participate in numerous protein regulatory processes. Abnormal deubiquitinase activity has been linked to cancer. In some embodiments, the deubiquitinase inhibitor is spautin-1.

In some embodiments, the compound is an ICAMI expression inhibitor. Intercellular adhesion molecule 1 (ICAM1), also known as CD45, is a glycoprotein involved in stabilizing intercellular interactions and is associated with inflammatory and immune responses. In some embodiments, ICAM1 expression inhibitor is A205804.

In some embodiments, the compound is a calcium channel blocker. Calcium channel blockers are widely used to treat hypertension in patients. Calcium channel blockers have also been used to treat other associated ailments such as angina and chest pains. In some embodiments, the calcium channel blocker is felodipine (also known as Plendil).

In some embodiments, the compound is a glucosylglucose. Glucosylglucose are disaccharides used as sweeteners and are further broken down in the body by various disaccharidases such as sucrase, lactase, and maltase. In some embodiments, the glucosylglucose is D-(+)-maltose.

In some embodiments, the compound is a trace amine-associated receptor antagonist. Trace amine-associated receptors (TAAR) bind trace amines that are metabolic derivatives of various amino acids. TAARs are therapeutic targets for various neurological and psychiatric disorders. In some embodiments, the trace amine-associated receptor antagonist is PPT.

In some embodiments, the compound is a G protein-coupled receptor agonist. G-protein-coupled receptors (GPCR) are a large group of cell surface receptor proteins involved in various signaling pathways. GPCRs are implicated in many inflammatory, metabolic, cardiac, and monogenic disorders and represent an important class of drug targets. In some embodiments, the G protein-couple receptor agonist is GW-1100.

In some embodiments, one or more of the compounds above may function as a dual inhibitor that targets more than one enzyme or receptor. For example, pexmetinib is known to be both a TIE inhibitor and a p38 MAPK inhibitor.

These compounds are all commercially available and also may be synthesized according to methods known in the art. The structures of the compounds are shown in Table 1.

TABLE 1

| Compound | Structure | IUPAC Name |
|---|---|---|
| ralimetinib (LY2228820) | 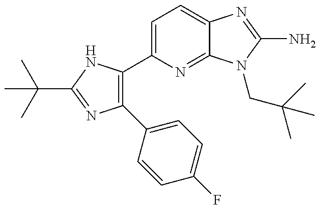 [SMILES: CC(C)(C)Cn1c(N)nc2ccc(-c3[nH]c(C(C)(C)C)nc3-c3ccc(F)cc3)nc21] | 5-[2-tert-butyl-4-(4-fluorophenyl)- 1H-imidazol-5-yl]-3-(2,2- dimethylpropyl)imidazo[4,5- b]pyridin-2-amine |
| talmapimod | 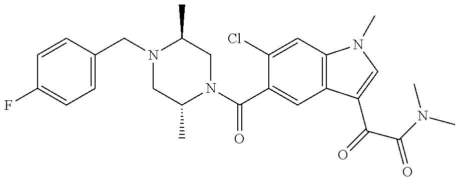 [SMILES: C[C@@H]1CN(Cc2ccc(F)cc2)[C@@H](C)CN1C(=O)c1cc2c(C(=O)C(=O)N(C)C)cn(C)c2cc1Cl] | 2-[6-chloro-5-[(2R,5S)-4-[(4- fluorophenyl)methyl]-2,5- dimethylpiperazine-1-carbonyl]-1- methylindol-3-yl]-N,N-dimethyl-2- oxoacetamide |
| SB-239063 | 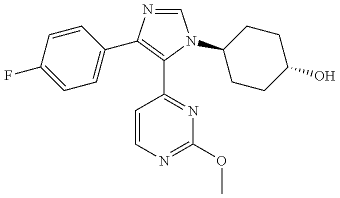 [SMILES: COc1nccc(-c2c(-c3ccc(F)cc3)ncn2[C@H]2CC[C@H](O)CC2)n1] | 4-[4-(4-fluorophenyl)-5-(2- methoxypyrimidin-4-yl)imidazol- 1-yl]cyclohexan-1-ol |
| neflamapimod (VX-745) | 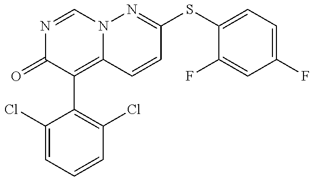 [SMILES: O=c1ncn2nc(Sc3ccc(F)cc3F)ccc2c1-c1c(Cl)cccc1Cl] | 5-(2,6-dichlorophenyl)-2-(2,4- difluorophenyl)sulfanylpyrimido[1, 6-b]pyridazin-6-one |
| SB-202190 | 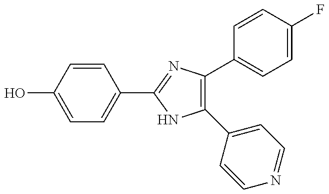 [SMILES: Oc1ccc(-c2nc(-c3ccc(F)cc3)c(-c3ccncc3)[nH]2)cc1] | 4-[4-(4-fluorophenyl)-5-pyridin-4- yl-1H-imidazol-2-yl]phenol |
| TA-01 | 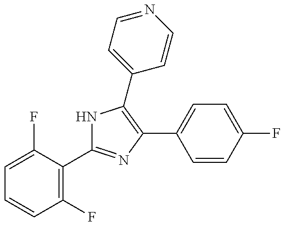 [SMILES: Fc1ccc(-c2nc(-c3c(F)cccc3F)[nH]c2-c2ccncc2)cc1] | 4-[2-(2,6-difluorophenyl)-4-(4- fluorophenyl)-1H-imidazol-5- yl]pyridine |
| SB 242235 | 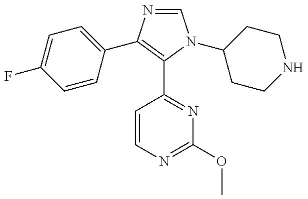 [SMILES: COc1nccc(-c2c(-c3ccc(F)cc3)ncn2C2CCNCC2)n1] | 4-[5-(4-fluorophenyl)-3-piperidin- 4-ylimidazol-4-yl]-2- methoxypyrimidine |
| AL-8697 | 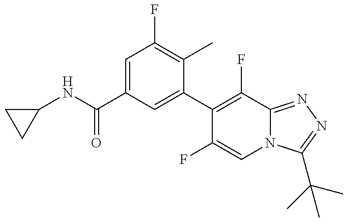 [SMILES: Cc1c(F)cc(C(=O)NC2CC2)cc1-c1c(F)cn2c(C(C)(C)C)nnc2c1F] | 3-(3-tert-butyl-6,8-difluoro- [1,2,4]triazolo[4,3-a]pyridin-7-yl)- N-cyclopropyl-5-fluoro-4- methylbenzamide |
| losmapimod | 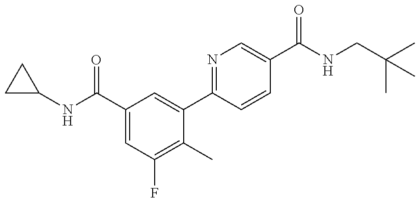 [SMILES: Cc1c(F)cc(C(=O)NC2CC2)cc1-c1ccc(C(=O)NCC(C)(C)C)cn1] | 6-[5-(cyclopropylcarbamoyl)-3- fluoro-2-methylphenyl]-N-(2,2- dimethylpropyl)pyridine-3- carboxamide |
| RepSox | 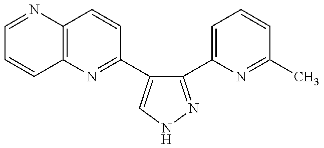 [SMILES: Cc1cccc(-c2n[nH]cc2-c2ccc3ncccc3n2)n1] | 2-[5-(6-methylpyridin-2-yl)-1H- pyrazol-4-yl]-1,5-naphthyridine |
| tovorafenib (TAK-580) | 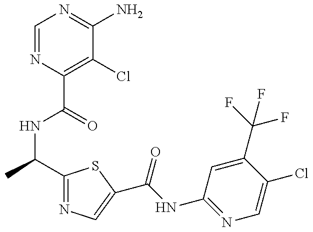 [SMILES: C[C@@H](NC(=O)c1ncnc(N)c1Cl)c1ncc(C(=O)Nc2cc(C(F)(F)F)c(Cl)cn2)s1] | 2-[(1R)-1-[(6-amino-5- chloropyrimidine-4- carbonyl)amino]ethyl]-N-[5- chloro-4-(trifluoromethyl)pyridin- 2-yl]-1,3-thiazole-5-carboxamide |
| FRAX486 | 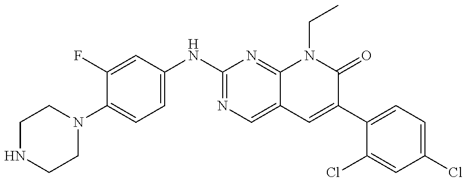 [SMILES: CCn1c(=O)c(-c2ccc(Cl)cc2Cl)cc2cnc(Nc3ccc(N4CCNCC4)c(F)c3)nc21] | 6-(2,4-dichlorophenyl)-8-ethyl-2- (3-fluoro-4-piperazin-1- ylanilino)pyrido[2,3-d]pyrimidin- 7-one |
| pexmetinib (ARRY-614) | 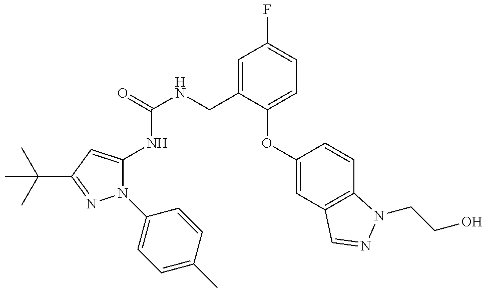 [SMILES: Cc1ccc(-n2nc(C(C)(C)C)cc2NC(=O)NCc2cc(F)ccc2Oc2ccc3c(cnn3CCO)c2)cc1] | tert-butyl-2-(4- methylphenyl)pyrazol-3-yl]-3-[[5- fluoro-2-[1-(2- hydroxyethyl)indazol-5- yl]oxyphenyl]methyl]urea |
| adezmapimod (SB-203580) | 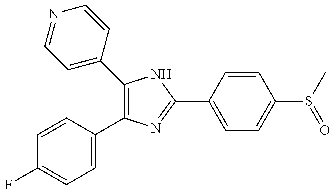 [SMILES: CS(=O)c1ccc(-c2nc(-c3ccc(F)cc3)c(-c3ccncc3)[nH]2)cc1] | 4-[4-(4-fluorophenyl)-2-(4- methylsulfinylphenyl)-1H- imidazol-5-yl]pyridine |
| AZD3965 | 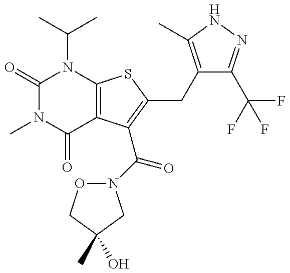 [SMILES: Cc1[nH]nc(C(F)(F)F)c1Cc1sc2c(c1C(=O)N1C[C@](C)(O)CO1)c(=O)n(C)c(=O)n2C(C)C] | 5-[(4S)-4-hydroxy-4-methyl-1,2- oxazolidine-2-carbonyl]-3-methyl- 6-[[5-methyl-3-(trifluoromethyl)- 1H-pyrazol-4-yl]methyl]-1-propan- 2-ylthieno[2,3-d]pyrimidine-2,4- dione |
| GW-1100 | 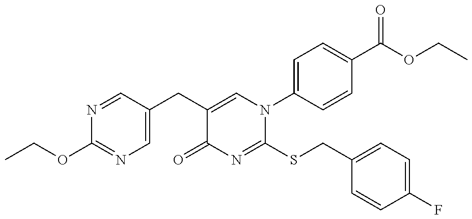 [SMILES: CCOC(=O)c1ccc(-n2cc(Cc3cnc(OCC)nc3)c(=O)nc2SCc2ccc(F)cc2)cc1] | ethyl 4-[5-[(2-ethoxypyrimidin-5- yl)methyl]-2-[(4- fluorophenyl)methylsulfanyl]-4- oxopyrimidin-1-yl]benzoate |
| propyl pyrazole triol (PPT) | 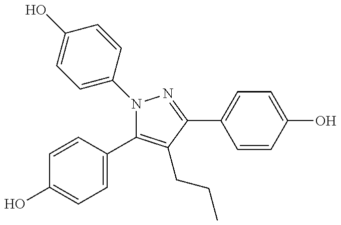 [SMILES: CCCc1c(-c2ccc(O)cc2)nn(-c2ccc(O)cc2)c1-c1ccc(O)cc1] | 4-[2,5-bis(4-hydroxyphenyl)-4- propylpyrazol-3-yl]phenol |
| RWJ-67657 | 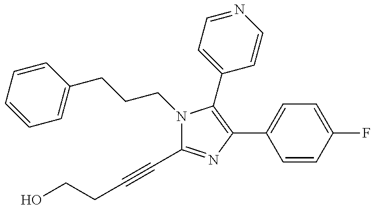 [SMILES: OCCC#Cc1nc(-c2ccc(F)cc2)c(-c2ccncc2)n1CCCc1ccccc1] | 4-[4-(4-fluorophenyl)-1-(3- phenylpropyl)-5-pyridin-4- ylimidazol-2-yl]but-3-yn-1-ol |
| SX 011 | 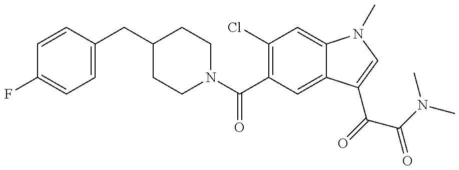 [SMILES: CN(C)C(=O)C(=O)c1cn(C)c2cc(Cl)c(C(=O)N3CCC(Cc4ccc(F)cc4)CC3)cc12] | 2-[6-chloro-5-[4-[(4- fluorophenyl)methyl]piperidine-1- carbonyl]-1-methylindol-3-yl]- N,N-dimethyl-2-oxoacetamide |
| felodipine (Plendil) | 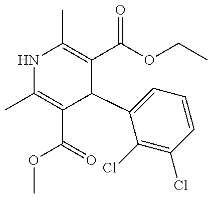 [SMILES: CCOC(=O)C1=C(C)NC(C)=C(C(=O)OC)C1c1cccc(Cl)c1Cl] | 5-O-ethyl 3-O-methyl 4-(2,3- dichlorophenyl)-2,6-dimethyl-1,4- dihydropyridine-3,5-dicarboxylate |
| SKF-86002 | 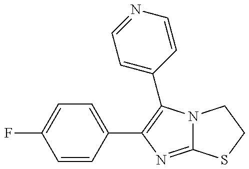 [SMILES: Fc1ccc(-c2nc3n(c2-c2ccncc2)CCS3)cc1] | 6-(4-fluorophenyl)-5-pyridin-4-yl- 2,3-dihydroimidazo[2,1- b][1,3]thiazole |
| D-(+)- MALTOSE | 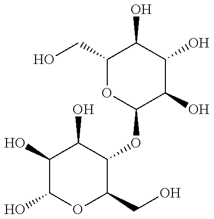 [SMILES: OC[C@H]1O[C@H](O[C@H]2[C@H](O)[C@H](O)[C@@H](O)O[C@@H]2CO)[C@H](O)[C@@H](O)[C@@H]1O] | (2R,3S,4S,5R,6R)-2- (hydroxymethyl)-6- [(2R,3S,4R,5R)-4,5,6-trihydroxy- 2-(hydroxymethyl)oxan-3- yl]oxyoxane-3,4,5-triol |
| TAK-715 | 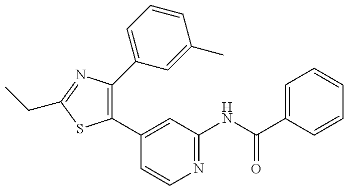 [SMILES: CCc1nc(-c2cccc(C)c2)c(-c2ccnc(NC(=O)c3ccccc3)c2)s1] | N-[4-[2-ethyl-4-(3-methylphenyl)- 1,3-thiazol-5-yl]pyridin-2- yl]benzamide |
| bis(maltolato) oxovanadium (IV) | 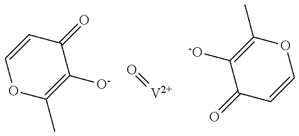 | 2-methyl-4-oxopyran-3- olate;oxovanadium(2+) |
| EO-1428 | 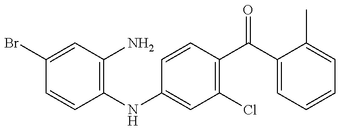 [SMILES: Cc1ccccc1C(=O)c1ccc(Nc2ccc(Br)cc2N)cc1Cl] | [4-(2-amino-4-bromoanilino)-2- chlorophenyl]-(2- methylphenyl)methanone |
| SM-164 | 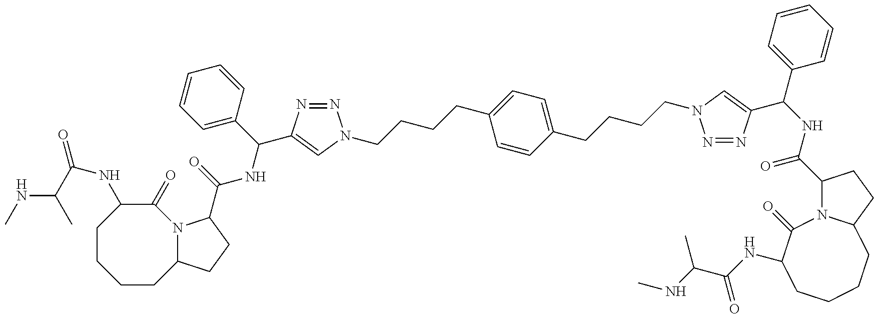 [SMILES: CNC(C)C(=O)NC1CCCCC2CCC(C(=O)NC(c3ccccc3)c3cn(CCCCc4ccc(CCCCn5cc(C(NC(=O)C6CCC7CCCCC(NC(=O)C(C)NC)C(=O)N76)c6ccccc6)nn5)cc4)nn3)N2C1=O] | (3S,6S, 10aS)-N-[(S)-[1-[4-[4-[4- [4-[(S)-[[(3S,6S,10aS)-6-[[(2S)-2- (methylamino)propanoyl]amino]-5- oxo-2,3,6,7,8,9,10,10a-octahydro- 1H-pyrrolo[1,2-a]azocine-3- carbonyl]amino]- phenylmethyl]triazol-1- yl]butyl]phenyl]butyl]triazol-4-yl]- phenylmethyl]-6-[(2S)-2- (methylamino)propanoyl]amino]-5- oxo-2,3,6,7,8,9,10,10a-octahydro- 1H-pyrrolo[1,2-a]azocine-3- carboxamide |
| BMS-265246 | 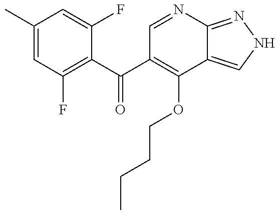 [SMILES: CCCCOc1c(C(=O)c2c(F)cc(C)cc2F)cnc2n[nH]cc12] | (4-butoxy-2H-pyrazolo[3,4- b]pyridin-5-yl)-(2,6-difluoro-4- methylphenyl)methanone |
| semapimod | 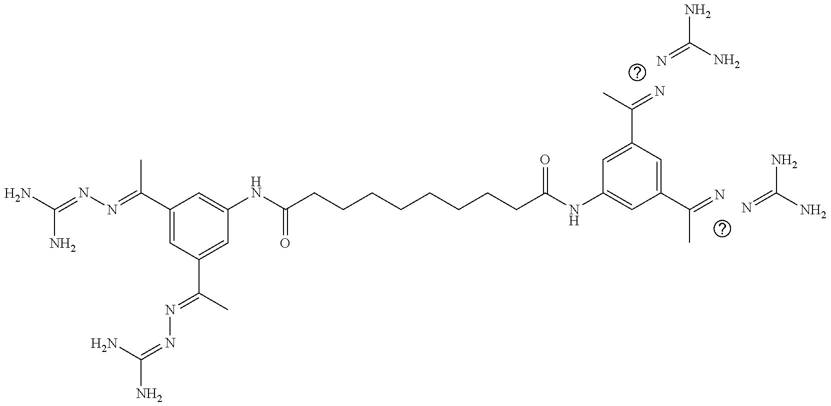 | N,N′-bis[3,5-bis[(E)-N- (diaminomethylideneamino)-C- methylcarbonimidoyl]phenyl] decanediamide |
| LY2109761 | 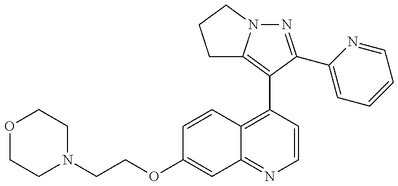 [SMILES: c1ccc(-c2nn3c(c2-c2ccnc4cc(OCCN5CCOCC5)ccc24)CCC3)nc1] | 4-[2-[4-(2-pyridin-2-yl-5,6- dihydro-4H-pyrrolo[1,2-b]pyrazol- 3-yl)quinolin-7- yl]oxyethyl]morpholin |
| GDC-0879 | 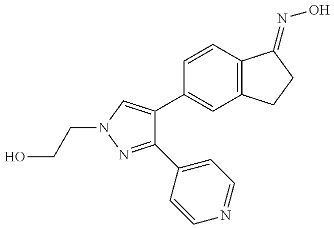 [SMILES: OCCn1cc(-c2ccc3c(c2)CC/C3=N\O)c(-c2ccncc2)n1] | 2-[4-[(1E)-1-hydroxyimino-2,3- dihydroinden-5-yl]-3-pyridin-4- ylpyrazol-1-yl]ethanol |
| PPY-A | 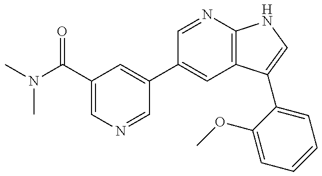 [SMILES: COc1ccccc1-c1c[nH]c2ncc(-c3cncc(C(=O)N(C)C)c3)cc12] | 5-[3-(2-methoxyphenyl)-1H- pyrrolo[2,3-b]pyridin-5-yl]-N,N- dimethylpyridine-3-carboxamide |
| K02288 | 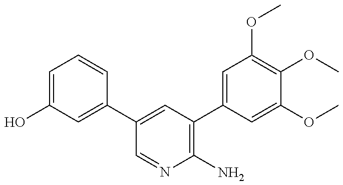 [SMILES: COc1cc(-c2cc(-c3cccc(O)c3)cnc2N)cc(OC)c1OC] | 3-[6-amino-5-(3,4,5- trimethoxyphenyl)pyridin-3- yl]phenol |
| WZ4003 | 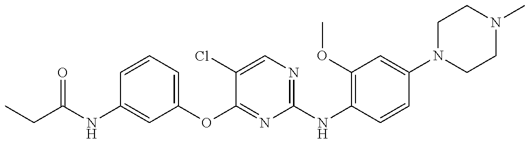 [SMILES: CCC(=O)Nc1cccc(Oc2nc(Nc3ccc(N4CCN(C)CC4)cc3OC)ncc2Cl)c1] | N-[3-[5-chloro-2-[2-methoxy-4-(4- methylpiperazin-1- yl)anilino]pyrimidin-4- yl]oxyphenyl]propanamide |
| SCH-51344 | 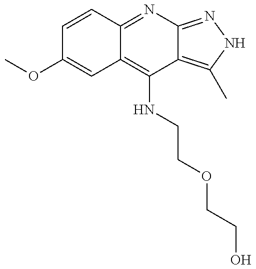 [SMILES: COc1ccc2nc3n[nH]c(C)c3c(NCCOCCO)c2c1] | 2-[2-[(6-methoxy-3-methyl-2H- pyrazolo[3,4-b]quinolin-4- yl)amino]ethox y]ethanol |
| ID-8 | 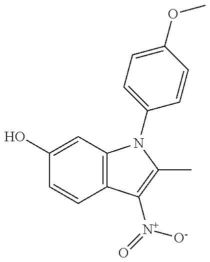 [SMILES: COc1ccc(-n2c(C)c([N+](=O)[O-])c3ccc(O)cc32)cc1] | 1-(4-methoxyphenyl)-2-methyl-3- nitroindol-6-ol |
| AZD5582 | 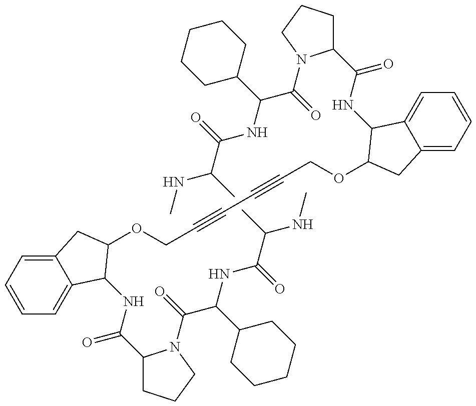 [SMILES: CNC(C)C(=O)NC(C(=O)N1CCCC1C(=O)NC1c2ccccc2CC1OCC#CC#CCOC1Cc2ccccc2C1NC(=O)C1CCCN1C(=O)C(NC(=O)C(C)NC)C1CCCCC1)C1CCCCC1] | (2S)-1-[(2S)-2-cyclohexyl-2- [[(2S)-2- (methylamino)propanoyl]amino] acetyl]-N-[(1S,2R)-2-[6-[(1S,2R)- 1-[[(2S)-1-[(2S)-2-cyclohexyl-2- [[(2S)-2- (methylamino)propanoyl]amino] acetyl]pyrrolidine-2- carbonyl]amino]-2,3-dihydro-1H- inden-2-yl]oxy]hexa-2,4-diynoxy]- 2,3-dihydro-1H-inden-1- yl]pyrrolidine-2-carboxamide |
| CUDC-427 | 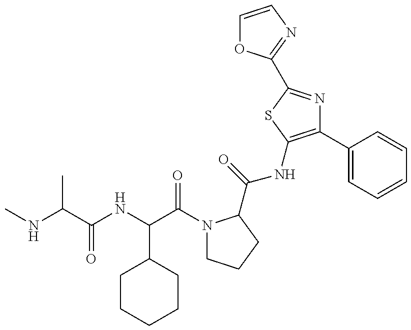 [SMILES: CNC(C)C(=O)NC(C(=O)N1CCCC1C(=O)Nc1sc(-c2ncco2)nc1-c1ccccc1)C1CCCCC1] | (2S)-1-[(2S)-2-cyclohexyl-2- [[(2S)-2- (methylamino)propanoyl]amino] acetyl]-N-[2-(1,3-oxazol-2-yl)-4- phenyl-1,3-thiazol-5- yl]pyrrolidine-2-carboxamide |
| spautin-1 | 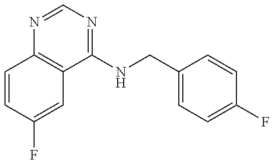 [SMILES: Fc1ccc(CNc2ncnc3ccc(F)cc23)cc1] | 6-fluoro-N-[(4- fluorophenyl)methyl]quinazolin-4- amine |
| A205804 | 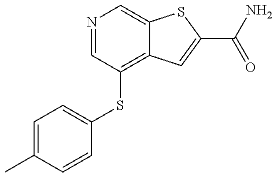 [SMILES: Cc1ccc(Sc2cncc3sc(C(N)=O)cc23)cc1] | 4-(4- methylphenyl)sulfanylthieno[2,3- c]pyridine-2-carboxamide |
| SB590885 | 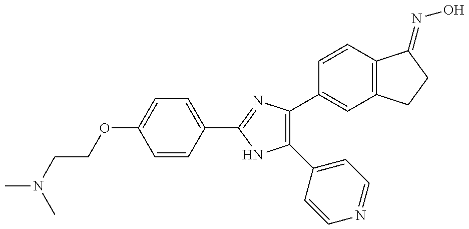 [SMILES: CN(C)CCOc1ccc(-c2nc(-c3ccc4c(c3)CC/C4=N\O)c(-c3ccncc3)[nH]2)cc1] | (NE)-N-[5-[2-[4-[2- (dimethylamino)ethoxy]phenyl]-5- pyridin-4-yl-1H-imidazol-4-yl]- 2,3-dihydroinden-1- ylidene]hydroxylamine |

In some embodiments, methods for treating Leigh syndrome, comprises administering a therapeutically effective amount of a compound which is:

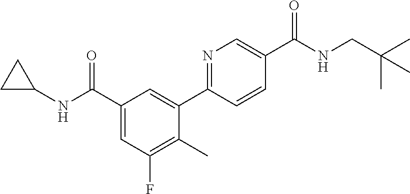 [SMILES: Cc1c(F)cc(C(=O)NC2CC2)cc1-c1ccc(C(=O)NCC(C)(C)C)cn1]

or a pharmaceutically acceptable salt thereof.

In some embodiments, methods for treating Leigh syndrome, comprises administering a therapeutically effective amount of a compound which is:

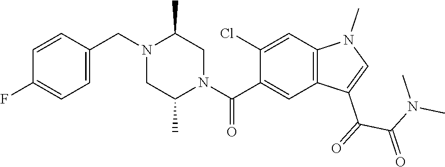 [SMILES: C[C@@H]1CN(Cc2ccc(F)cc2)[C@@H](C)CN1C(=O)c1cc2c(C(=O)C(=O)N(C)C)cn(C)c2cc1Cl]

or a pharmaceutically acceptable salt thereof.

In some embodiments, methods for treating Leigh syndrome, comprises administering a therapeutically effective amount of a compound which is:

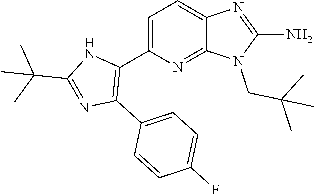 [SMILES: CC(C)(C)Cn1c(N)nc2ccc(-c3[nH]c(C(C)(C)C)nc3-c3ccc(F)cc3)nc21]

or a pharmaceutically acceptable salt thereof.

In some embodiments, methods for treating Leigh syndrome, comprises administering a therapeutically effective amount of a compound which is:

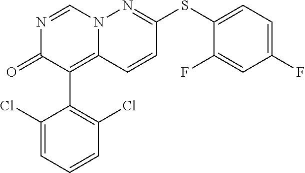 [SMILES: O=c1ncn2nc(Sc3ccc(F)cc3F)ccc2c1-c1c(Cl)cccc1Cl]

or a pharmaceutically acceptable salt thereof.

Also provided herein, in some embodiments, are pharmaceutical compositions that comprise one or more of the compounds described herein, or a pharmaceutically acceptable salt thereof, and one or more pharmaceutically acceptable vehicles selected from carriers, adjuvants and excipients. Suitable pharmaceutically acceptable vehicles may include, for example, inert solid diluents and fillers, diluents, including sterile aqueous solution and various organic solvents, permeation enhancers, solubilizers and adjuvants. Such compositions are prepared in a manner well known in the pharmaceutical art. See, e.g., Remington's Pharmaceutical Sciences, Mace Publishing Co., Philadelphia, Pa. 17th Ed. (1985); and Modern Pharmaceutics, Marcel Dekker, Inc. 3rd Ed. (G.S. Banker & C.T. Rhodes, Eds.).

The pharmaceutical compositions may be administered in either single or multiple doses. The pharmaceutical composition may be administered by various methods including, for example, rectal, buccal, intranasal, and transdermal routes. In certain embodiments, the pharmaceutical composition may be administered by intra-arterial injection, intravenously, intraperitoneally, parenterally, intramuscularly, subcutaneously, orally, topically, or as an inhalant.

One mode for administration is parenteral, for example, by injection. The forms in which the pharmaceutical compositions described herein may be incorporated for administration by injection include, for example, aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Oral administration may be another route for administration of the compounds described herein. Administration may be via, for example, capsule or enteric coated tablets. In making the pharmaceutical compositions that include at least one compound described herein or a pharmaceutically acceptable salt, isotopically enriched analog, stereoisomer, mixture of stereoisomers, or prodrug thereof, the active ingredient is usually diluted by an excipient and/or enclosed within such a carrier that can be in the form of a capsule, sachet, paper, or other container. When the excipient serves as a diluent, it can be in the form of a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, sterile injectable solutions, and sterile packaged powders.

Some examples of suitable excipients include, e.g., lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The formulations can additionally include lubricating agents such as talc, magnesium stearate, and mineral oil; wetting agents; emulsifying and suspending agents; preserving agents such as methyl and propylhydroxy-benzoates; sweetening agents; and flavoring agents.

The compositions that include at least one compound described herein or a pharmaceutically acceptable salt, isotopically enriched analog, stereoisomer, mixture of stereoisomers, or prodrug thereof can be formulated so as to provide quick, sustained or delayed release of the active ingredient after administration to the subject by employing procedures known in the art. Controlled release drug delivery systems for oral administration include osmotic pump systems and dissolutional systems containing polymer-coated reservoirs or drug-polymer matrix formulations. Another formulation for use in the methods disclosed herein employ transdermal delivery devices (“patches”). Such transdermal patches may be used to provide continuous or discontinuous infusion of the compounds described herein in controlled amounts. The construction and use of transdermal patches for the delivery of pharmaceutical agents is well known in the art. Such patches may be constructed for continuous, pulsatile, or on demand delivery of pharmaceutical agents.

For preparing solid compositions such as tablets, the principal active ingredient may be mixed with a pharmaceutical excipient to form a solid preformulation composition containing a homogeneous mixture of a compound described herein or a pharmaceutically acceptable salt, isotopically enriched analog, stereoisomer, mixture of stereoisomers, or prodrug thereof. When referring to these preformulation compositions as homogeneous, the active ingredient may be dispersed evenly throughout the composition so that the composition may be readily subdivided into equally effective unit dosage forms such as tablets, pills, and capsules.

The tablets or pills of the compounds described herein may be coated or otherwise compounded to provide a dosage form affording the advantage of prolonged action, or to protect from the acid conditions of the stomach. For example, the tablet or pill can include an inner dosage and an outer dosage component, the latter being in the form of an envelope over the former. The two components can be separated by an enteric layer that serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

Compositions for inhalation or insufflation may include solutions and suspensions in pharmaceutically acceptable, aqueous, or organic solvents, or mixtures thereof, and powders. The liquid or solid compositions may contain suitable pharmaceutically acceptable excipients as described herein. In certain embodiments, the compositions are administered by the oral or nasal respiratory route for local or systemic effect. In other embodiments, compositions in pharmaceutically acceptable solvents may be nebulized by use of inert gases. Nebulized solutions may be inhaled directly from the nebulizing device or the nebulizing device may be attached to a facemask tent, or intermittent positive pressure breathing machine. Solution, suspension, or powder compositions may be administered, in one embodiment, orally or nasally, from devices that deliver the formulation in an appropriate manner.

The amount of the compound in a pharmaceutical composition or formulation can vary within the full range employed by those skilled in the art. Typically, the formulation will contain, on a weight percent (wt %) basis, from about 0.01-99.99 wt % of a compound of this disclosure based on the total formulation, with the balance being one or more suitable pharmaceutical excipients. In one embodiment, the compound is present at a level of about 1-80 wt %. Representative pharmaceutical formulations are described below.

### Formulation Example 1—Tablet formulation

The following ingredients are mixed intimately and pressed into single scored tablets.

| | Quantity per |
|---|---|
| Ingredient | tablet, mg |
| compound of this disclosure | 400 |
| Cornstarch | 50 |
| croscarmellose sodium | 25 |
| Lactose | 120 |
| magnesium stearate | 5 |

### Formulation Example 2—Capsule formulation

The following ingredients are mixed intimately and loaded into a hard-shell gelatin capsule.

| | Quantity per |
|---|---|
| Ingredient | capsule, mg |
| compound of this disclosure | 200 |
| lactose, spray-dried | 148 |
| magnesium stearate | 2 |

### Formulation Example 3—Suspension formulation

The following ingredients are mixed to form a suspension for oral administration.

| Ingredient | Amount | |
|---|---|---|
| compound of this disclosure | 1.0 | g |
| fumaric acid | 0.5 | g |
| sodium chloride | 2.0 | g |
| methyl paraben | 0.15 | g |
| propyl paraben | 0.05 | g |
| granulated sugar | 25.0 | g |
| sorbitol (70% solution) | 13.00 | g |
| Veegum K (Vanderbilt Co.) | 1.0 | g |
| Flavoring | 0.035 | mL |
| Colorings | 0.5 | mg |

| | |
|---|---|
| distilled water | q.s. to 100 mL |

### Formulation Example 4—Injectable formulation

The following ingredients are mixed to form an injectable formulation.

| Ingredient | Amount |
|---|---|
| compound of this disclosure | 0.2 mg-20 mg |
| sodium acetate buffer solution, 0.4M | 2.0 mL |
| HC1 (1N) or NaOH (1N) | q.s. to suitable pH |
| water (distilled, sterile) | q.s. to 20 mL |

### Formulation Example 5—Suppository Formulation

A suppository of total weight 2.5 g is prepared by mixing the compound of this disclosure with Witepsol® H-15 (triglycerides of saturated vegetable fatty acid; Riches-Nelson, Inc., New York), and has the following composition:

| Ingredient | Amount |
|---|---|
| Compound of this disclosure | 500 mg |
| Witepsol ® H-15 | balance |

### Kits

Provided herein are also kits that include a compound of the disclosure and suitable packaging. In one embodiment, a kit further includes instructions for use. In one aspect, a kit includes a compound of the disclosure and a label and/or instructions for use of the compounds in the treatment of the indications, including the diseases or conditions, described herein.

Provided herein are also articles of manufacture that include a compound described herein in a suitable container. The container may be a vial, jar, ampoule, preloaded syringe, and intravenous bag.

### Dosing

The specific dose level of a compound of the present disclosure for any particular subject will depend upon a variety of factors including the activity of the specific compound employed, the age, body weight, general health, sex, diet, time of administration, route of administration, and rate of excretion, drug combination and the severity of the particular disease in the subject undergoing therapy. For example, a dosage may be expressed as a number of milligrams of a compound described herein per kilogram of the subject's body weight (mg/kg). Dosages of between about 0.1 and 150 mg/kg may be appropriate. In some embodiments, about 0.1 and 100 mg/kg may be appropriate. In other embodiments a dosage of between 0.5 and 60 mg/kg may be appropriate. Normalizing according to the subject's body weight is particularly useful when adjusting dosages between subjects of widely disparate size, such as occurs when using the drug in both children and adult humans or when converting an effective dosage in a non-human subject such as dog to a dosage suitable for a human subject.

The daily dosage may also be described as a total amount of a compound described herein administered per dose or per day. Daily dosage of a compound described herein may be between about 1 mg and 4,000 mg, between about 2,000 to 4,000 mg/day, between about 1 to 2,000 mg/day, between about 1 to 1,000 mg/day, between about 10 to 500 mg/day, between about 20 to 500 mg/day, between about 50 to 300 mg/day, between about 75 to 200 mg/day, or between about 15 to 150 mg/day.

When administered orally, the total daily dosage for a human subject may be between 1 mg and 1,000 mg, between about 1,000-2,000 mg/day, between about 10-500 mg/day, between about 50-300 mg/day, between about 75-200 mg/day, or between about 100-150 mg/day.

The compounds of the present disclosure or the compositions thereof may be administered once, twice, three, or four times daily, using any suitable mode described above. Also, administration or treatment with the compounds may be continued for a number of days; for example, commonly treatment would continue for at least 7 days, 14 days, or 28 days, for one cycle of treatment. Treatment cycles are well known, and are frequently alternated with resting periods of about 1 to 28 days, commonly about 7 days or about 14 days, between cycles. The treatment cycles, in other embodiments, may also be continuous.

In a particular embodiment, the method comprises administering to the subject an initial daily dose of about 1 to 800 mg of a compound described herein and increasing the dose by increments until clinical efficacy is achieved. Increments of about 5, 10, 25, 50, or 100 mg can be used to increase the dose. The dosage can be increased daily, every other day, twice per week, or once per week.

### EXAMPLES

The following examples are included to demonstrate specific embodiments of the disclosure. It should be appreciated by those of skill in the art that the techniques disclosed in the examples which follow represent techniques to function well in the practice of the disclosure, and thus can be considered to constitute specific modes for its practice. However, those of skill in the art should, in light of the present disclosure, appreciate that many changes can be made in the specific embodiments which are disclosed and still obtain a like or similar result without departing from the spirit and scope of the disclosure.

This disclosure is further understood by reference to the following examples, which are intended to be purely exemplary of this disclosure. This disclosure is not limited in scope by the exemplified embodiments, which are intended as illustrations of single aspects of this disclosure only. Any methods that are functionally equivalent are within the scope of this disclosure. Various modifications of this disclosure in addition to those described herein will become apparent to those skilled in the art from the foregoing description and accompanying figures. Such modifications fall within the scope of the appended claims.

In the specification and in the examples below, all temperatures are in degrees Celsius. In addition, the following abbreviations have the following meanings. If not defined, these abbreviations have their art recognized meaning.

ECHS1 is active in several metabolic pathways in the mitochondria. In particular, ECHS1 catalyzes the second step in the beta-oxidation pathway of fatty acid metabolism, breaking down fatty acids into acetyl-CoA. ECHS1 enzyme activity may be determined by a cell viability assay in a fatty acid-enriched medium and/or glucose-depleted medium. In this assay, cells affected with ECHS1 mutation are unable to metabolize fatty acids in the altered medium and therefore will be less viable than normal cells. Consequently, a compound that modulates ECHS1 activity, when administered, increases cell viability by restoring enzymatic activity and enabling fatty acid metabolism. The cell viability assay as described above may be used to effectively screen for compounds that modulate ECHSI activity.

Initial cell viability experiments in galactose-and propionate-enriched media showed promise in identifying potential candidates that may modulate ECHS1 activity. In the galactose and propionate screening assays described below, ECHS1 fibroblasts were treated with compounds from the Prestwick/Selleck Chem and SPECS library in the presence of galactose-or propionate-enriched media and evaluated for survivability. A high cell count indicates efficacy of the particular compound in rescuing ECHS1 activity by restoring fatty acid metabolism.

### Example 1: Galactose screening assay

Galactose Screen: Compound library plates were obtained from Prestwick/Selleck and SPECS (7,591 compounds total, 27 compounds/plate/concentration at 1 μM and 10 μM, 384-well plates). ECHS1 Fibroblast cells were harvested at 95% confluent with trypsin and suspended in Dulbecco's Modified Eagle Medium (DMEM)+10% Fetal Bovine Serum (FBS). The suspension was centrifuged at 200x g for 5 min. The suspension was discarded and the pellet was resuspended in the 5.5 mM galactose medium (DMEM+10% FBS+5.5 mM galactose) and seeded at 750 cells/well (50 μL) into the compound wells (1 μM and 10 μM). For the positive control experiment, the cells were suspended in 5.5 mM galactose medium+5.5 mM glucose+0.01% DMSO and seeded at 750 cells/well (50 μL) into the blank wells. For the negative control experiment, the cells were suspended in the 5.5 mM galactose medium+0.01% DMSO and seeded at 750 cells/well (50 μL) into the blank wells. The seeded plates were incubated at 37° C. for 96 hours, then 15 μL/well of 16% paraformaldehyde solution was added to the plates and allowed to sit for 15 min to fix the cells. The plates were then washed 3x with 50 μL/well of phosphate-buffered saline (PBS). The cells were stained with 25 pL/well of a solution of 4,6-diamidino-2-phenylindole dilactate (DAPI, Invitrogen) and left at room temperature for 1 hr. The plates were then washed 3x with 50 μL/well of PBS prior to imaging. Agilent BioTek EL406 Microplate Washer Dispenser was used for the cell fixation and staining protocol. The plates were imaged using GE InCell2200 Cell Analyzer using PerkinElmer Columbus software quantification protocol. The data was analyzed using Genedata, Vortex, and Abase software. A Z-score value of >2 was used for the Prestwick/Selleck compound library and >3 for the SPECS library (based on the negative control population) to determine hit selection.

A high cell count relative to the control experiment indicated enhanced cell survivability in the galactose-enriched medium, demonstrating that a particular compound is effective in modulating ECHS1 enzyme activity by restoring fatty acid metabolism (in this case galactose). In the galactose screen, a total of 7 compounds at 1 μM, 12 compounds at 10 μM, and 1 compound at both concentrations were identified from the Prestwick/Selleck library. A total of 53 compounds at 1 uM, 47 compounds at 10 μM, and 27 compounds from both concentrations were identified from the SPECS library. These compounds were selected for further dosage-dependent screening as shown in Example 3.

### Example 2: Propionate Screening Assay

Propionate Screen: ECHS1 Fibroblast cells were harvested at 95% confluent with trypsin and suspended in DMEM+10% FBS. The suspension was centrifuged at 200x g for 5 min. The suspension was discarded and the pellet was resuspended in the 10 mM propionate medium (DMEM+10% FBS+10 mM sodium propionate) and seeded at 750 cells/well (50 μL) into the compound wells (1 μM and 10 μM). For the positive control experiment, the cells were suspended in 10 mM propionate medium +5.5 mM glucose +0.01% DMSO and seeded at 750 cells/well (50 μL) into the blank wells. For the negative control experiment, the cells were suspended in the 10 mM propionate medium +0.01% DMSO and seeded at 750 cells/well (50 μL) into the blank wells. The seeded plates were incubated at 37° C. for 96 hours, then 15 μL/well of 16% paraformaldehyde solution was added to the plates and allowed to sit for 15 min to fix the cells. The plates were then washed 3x with 50 uL/well of PBS. The cells were stained with 25 μL/well of a solution DAPI and left at room temperature for 1 hr. The plates were then washed 3x with 50 uL/well of PBS prior to imaging. Agilent BioTek EL406 Microplate Washer Dispenser was used for the cell fixation and staining protocol. The plates were imaged using GE InCell2200 Cell Analyzer using PerkinElmer Columbus software quantification protocol. The data was analyzed using Genedata, Vortex, and Abase software. A Z-score value of >2 was used for both the Prestwick/Selleck and SPECS library (based on the negative control population) to determine hit selection.

In the propionate screen, a total of 3 compounds at 1 μM, 6 compounds at 10 μM, and 0 compound at both concentrations were identified from the Prestwick/Selleck library. A total of 37 compounds at 1 μM, 35 compounds at 10 μM, and 10 compounds from both concentrations were identified from the SPECS library. These compounds were selected for further dosage-dependent screening as shown in Example 3.

### Example 3: Potency Screen

A potency screen was further carried out on the initial hits identified in Examples 1 and 2. The purpose of the potency screen was to assess the dose-dependent response of each of the compounds and determine which compounds show the highest efficacy.

112 compounds total were identified from the galactose and propionate screening experiments and further evaluated in the potency screen. ECHS1 Fibroblast cells were harvested at 70-80% confluent with trypsin and suspended in DMEM+10% FBS. The suspension was centrifuged at 200x g for 5 min. The suspension was discarded and the pellet was resuspended in the either the 5.5 mM galactose medium (DMEM+10% FBS+5.5 mM of galactose) or the 10 mM propionate medium (DMEM+10% FBS+10 mM sodium propionate). The cells were seeded at 750 cells/well (50 μL) into a 384-well plate containing the test compounds (3-fold serial dilution across 10 plates, starting at 10 uM). For the positive control experiment, the cells were suspended in either the 5.5 mM galactose medium or 10 mM propionate medium+5.5 mM glucose+0.01% DMSO and seeded at 750 cells/well (50 μL) into the blank wells. For the negative control experiment, the cells were suspended in the 5.5 mM galactose or 10 mM propionate medium +0.01% DMSO and seeded at 750 cells/well (50 μL) into the blank wells. The seeded plates were incubated at 37° C. for 96 hours, then 15 μL/well of 16% paraformaldehyde solution was added to the plates and allowed to sit for 15 min to fix the cells. The plates were then washed 3x with 50 μL/well of PBS. The cells were stained with 25 μL/well of a solution of DAPI and left at room temperature for 1 hr. The plates were then washed 3x with 50 μL/well of PBS prior to imaging. Agilent BioTek EL406 Microplate Washer Dispenser was used for the cell fixation and staining protocol. The plates were imaged using GE InCell2200 Cell Analyzer using PerkinElmer Columbus software quantification protocol. The data was analyzed using Genedata, Vortex, and Abase software. A Z-score value of >2 was used for both the Prestwick/Selleck and SPECS library (based on the negative control population) to determine hit selection.

Of the 112 compounds tested, 26 hits were identified from the galactose assay and 18 hits were identified from the propionate assay. Three compounds (pexmetinib, TA-01, and RepSox) were identified as hits from both the galactose and propionate potency screens. The results are summarized in Table 2.

TABLE 2

| Galactose assay hits | Propionate assay hits |
|---|---|
| ralimetinib (LY2228820) | SM-164 |
| talmapimod | BMS-265246 |
| SB-239063 | pexmetinib (ARRY-614) |
| neflamapimod (VX-745) | semapimod |
| SB-202190 | LY2109761 |
| TA-01 | GDC-0879 |
| SB-242235 | PPY-A |
| AL-8697 | K02288 |
| losmapimod | WZ4003 |
| RepSox | SCH-51344 |
| tovorafenib (TAK-580) | ID-8 |
| FRAX486 | AZD5582 |
| pexmetinib (ARRY-614) | TA-01 |
| adezmapimod (SB-203580) | CUDC-427 |
| AZD3965 | spautin-1 |
| GW-1100 | RepSox |
| PPT | A205804 |
| RWJ-67657 | SB590885 |
| SX-011 | |
| felodipine (Plendil) | |
| D-(+)-maltose | |
| SKF-86002 | |
| TAK-715 | |
| bis(maltolato)oxovanadium(IV) | |
| EO-1428 | |

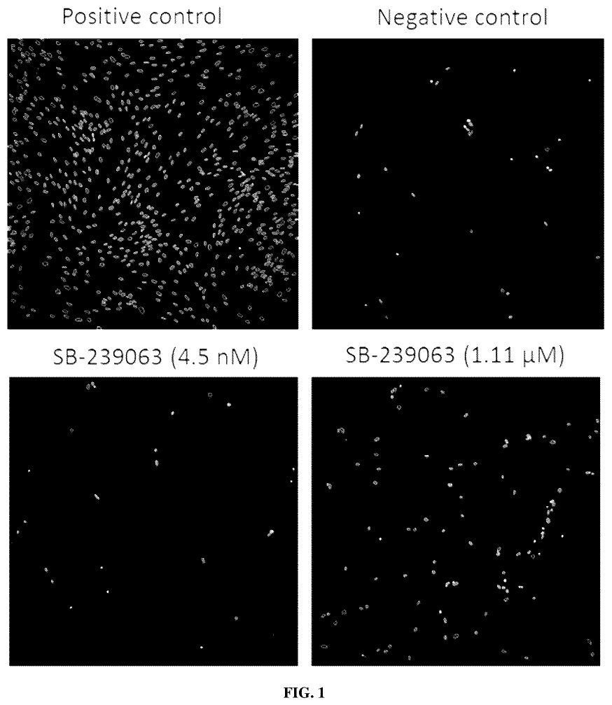

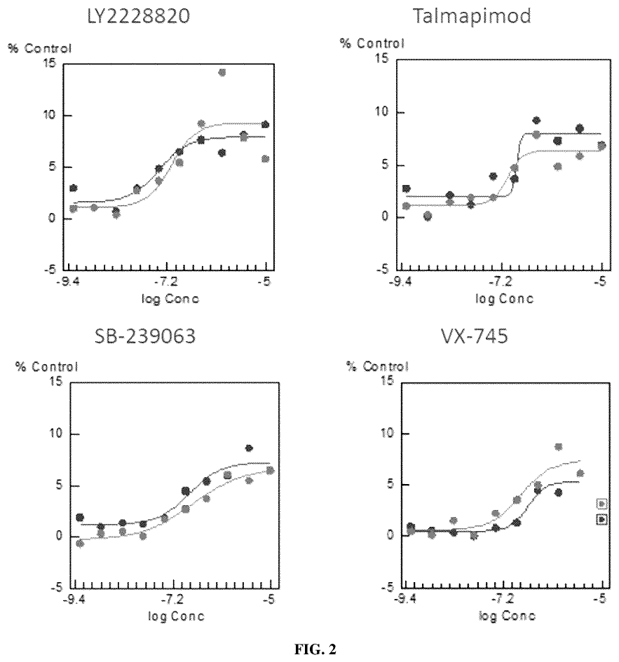

FIG. 1 shows the DAPI-stained raw nuclei image of cells treated with compound SB-239063 from the galactose potency screen, shown alongside the positive and negative control experiments. A concentration-dependent increase in nuclei count was observed for compound SB-239063 in the galactose assay. FIG. 2 shows the concentration-dependent curves of LY2228820, talmapimod, SB-239063, and VX-745 from the galactose potency screen showing a non-linear response.

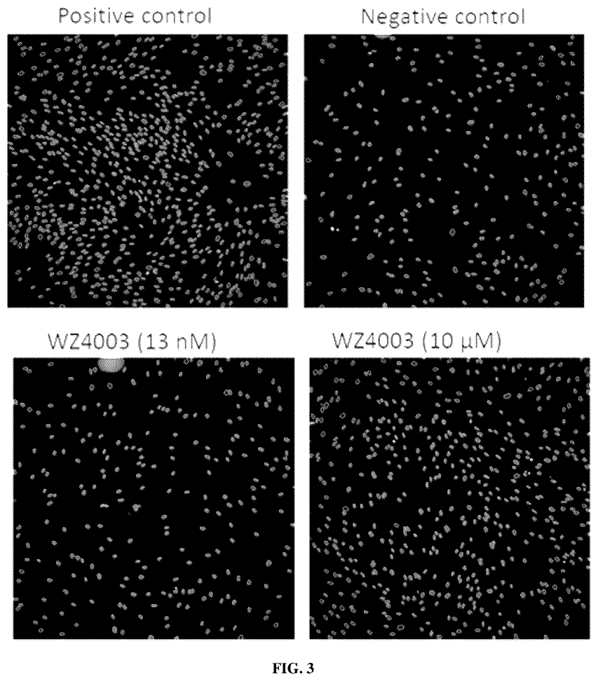

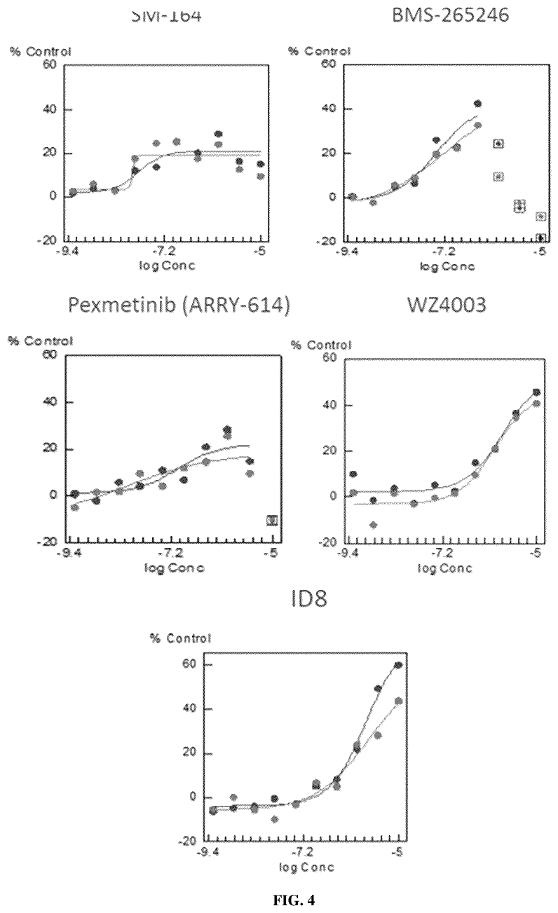

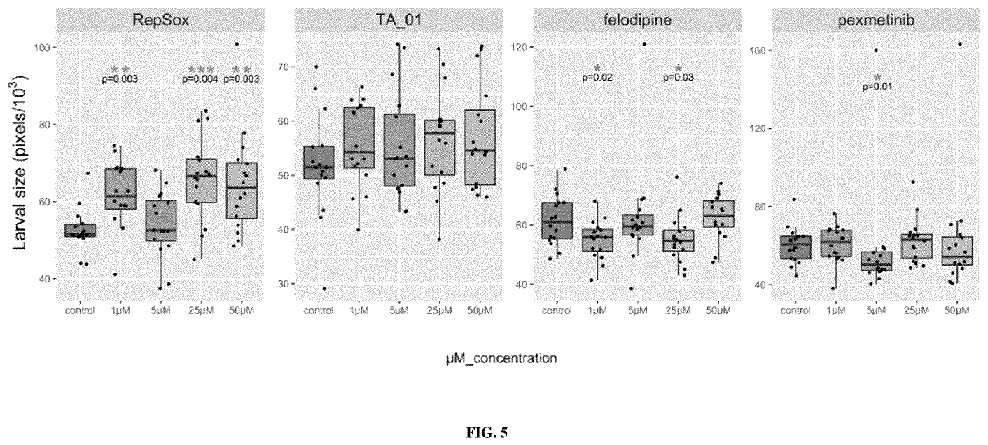

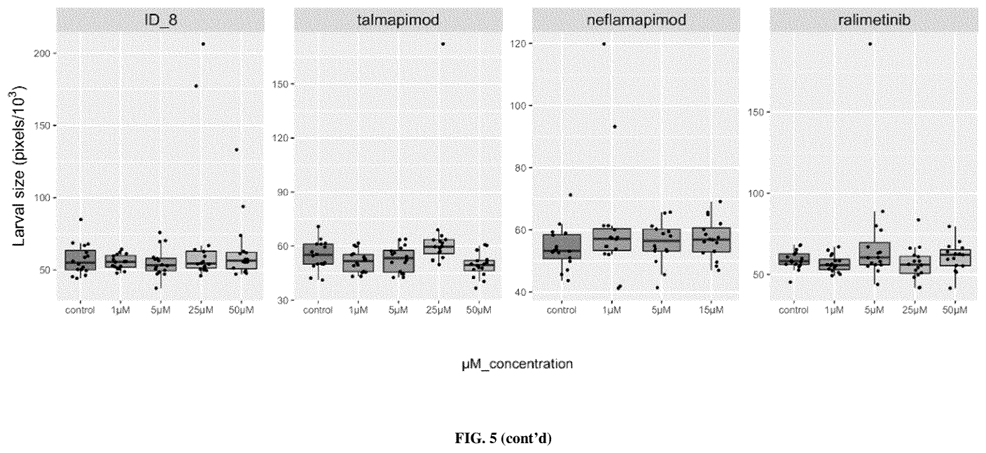

FIG. 3 shows the DAPI-stained raw nuclei image of cells treated with compound WZ4003 from the propionate potency screen, shown alongside the positive and negative control experiments. A concentration-dependent increase in nuclei count was observed for compound WZ4003 in the propionate assay. FIG. 4 shows the concentration-dependent curves of SM-164, BMS-265246, pexmetinib, WZ4003, and ID-8 from the propionate potency screen showing a non-linear response.

### Example 4: Fly Larva Experiment

To determine whether the Drosophila larvae experiment would be appropriate for further evaluation of compounds, the phenotype induced by ECHSI RNAi was identified. Ubiquitous knockdown of ECHS1 resulted in larvae that are significantly smaller than controls. It was noted that when extracted from food and placed in 1X PBS, ECHS1 knockdown larvae were buoyant and sank only ˜30% of the time, while age-matched control larvae always sank at 100% frequency. Of note, younger, size matched small control larvae also always sank, suggesting that the increased buoyancy of ECHS1 larvae was not caused by size alone.

To test the ability of drug compounds to rescue these two ECHS1 knockdown phenotypes, the larvae were fed with fly food with various concentrations of 10 candidate drugs and first measured for larval size. During the 6 days prior to collection and phenotyping, larvae were raised in the drug food and exposed to drug continuously throughout development. When larvae were collected and sampled for image analysis, it was found that all 10 compounds were able to increase mean size of the ECHS1 knockdown larvae. Independently, losmapimod, talmapimod, ralimetinib, and neflamapimod were selected at multiple doses and fed to larvae and the population-wide float/sink proportions were assayed. In contrast to untreated ECHS1 knockdown larvae, where 30% sank, treatment with drug compounds nearly doubled the sinking proportions to 60%. Considering the effect of ECHSI on buoyancy in light of its enzymatic function catalyzing the second step of mitochondrial fatty acid beta-oxidation, decreased ECHS1 activity was expected to increase levels of non-metabolized lipid which would increase buoyancy. Thus, compounds were identified that suppressed both quantitative and nominal phenotypes, one of which appears to be directly relevant to the enzymatic function of ECHS1.

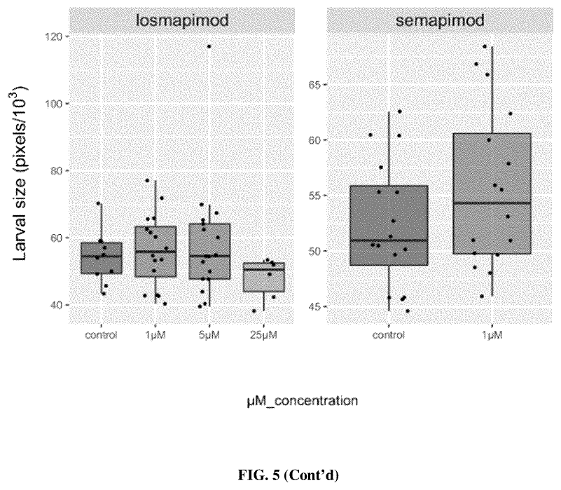

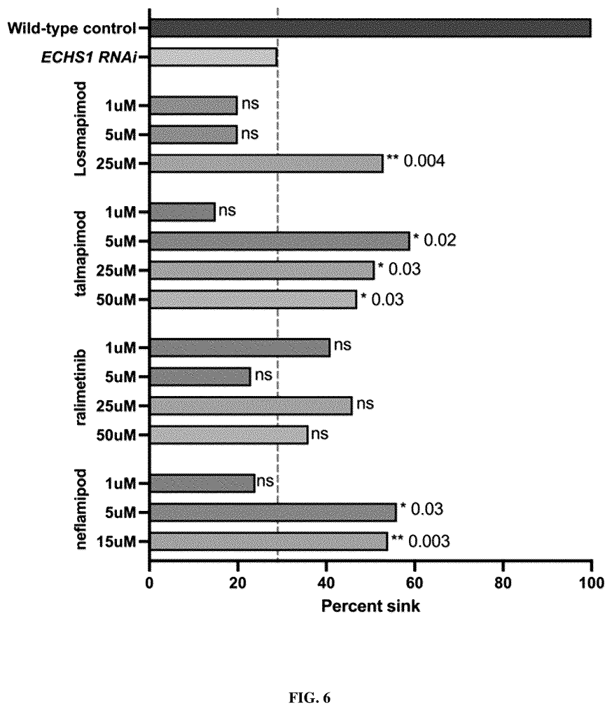

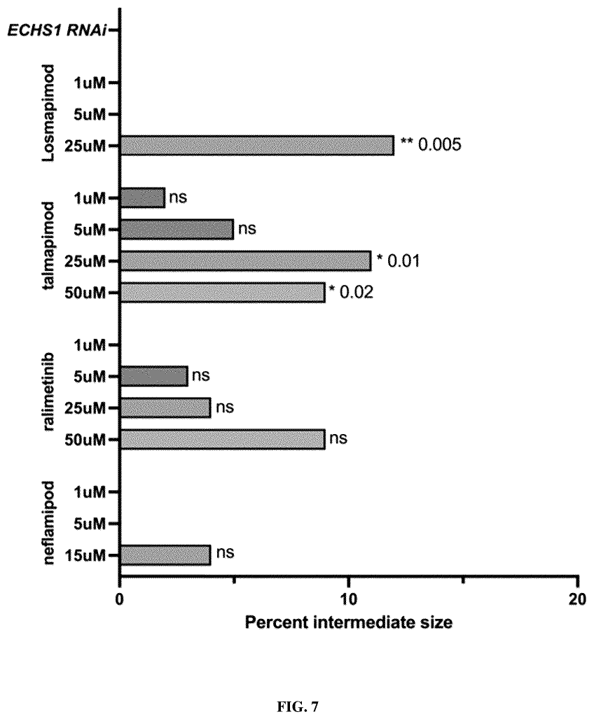

FIG. 5 shows the larval size increase for the compounds that were tested. FIG. 6 shows the effect of losmapimod, talmapimod, ralimetinib, and Neflamipod on ECHS1 RNAi buoyancy phenotype. FIG. 7 shows the effect on ECHS1 RNAi small size phenotype.

### Example 5: Mitochondrial Respiration Assay

To assess the mechanism of action of hit compounds from the screens of previous Examples, which were based on rescue of viability after metabolic challenge, Seahorse assay was used to measure mitochondrial function in response to treatment at multiple concentrations. The Seahorse assay measures the following parameters: oxygen consumption; basal respiration rate; ATP-linked respiration; maximal respiration; reserve capacity; and ATP production rate. At baseline (no treatment), the ECHS1 patient-derived fibroblasts had lower respiration rates than the control fibroblasts in glucose culturing conditions (20% reduction) and in galactose culturing conditions (30% reduction). The ECHS1 patient-derived fibroblasts did not adapt as well to galactose culturing compared to the control fibroblasts.

ECHS1 patient-derived fibroblasts and age-matched and sex-matched control fibroblasts were initially grown in standard media (DMEM+2 mM glutamine+1 mM pyruvate) containing 10 mM glucose as the energy source. Cells were seeded into specialized 96-well plates at a density of 10,000 cells per well. One day after plating, cells were treated with the test compounds at the concentrations of 0, 1, 5, or 10 μM. Half of the samples were cultured in 10 mM glucose media in addition to the test compounds. The remaining half of the sample were cultured in 10 mM galactose media, which is a metabolic challenge, in addition to the test compound. Test compounds were incubated with cells for 48 hours. According to the standard Seahorse protocol, the following stressors were added to cells in sequential order: 2 μM oligomycin; 5 μM carbonyl cyanide-4-(trifluoromethoxy)phenylhydrazone (FCCP); 9 μM FCCP; 2 μM rotenone and 2 μM antimycin A. Data were normalized to cell number per well using a Hoechst stain for cell nuclei. Two rounds of experiments were conducted. The results were consistent between the two rounds.

The following six test compounds were assessed: losmapimod, RepSox, pexmetinib, felodipine, ralimetinib and talmapimod with RepSox and ralimetinib only tested in the first round.

Treatment with losmapimod for 48 hours did not result in major changes to respiration in either glucose or galactose conditions. Treatment with RepSox for 48 hours did not result in major changes to respiration in either glucose or galactose conditions.

Treatment with felodipine for 48 hours did not result in major changes to respiration in either glucose or galactose conditions. However, felodipine appears to cause some impairment in respiration as evidenced by decreased basal and maximal respiration, which is compensated for by increased ATP production from glycolysis. Treatment with ralimetinib for 48 hours did not result in major changes to respiration in either glucose or galactose conditions.

Treatment with pexmetinib at 1 μM concentration for 48 hours increased cell number per well in both galactose and glucose cultured plates by 14%. In glucose-treated cells but less pronounced in galactose-treated cells, maximal respiration was increased to similar levels to the control cells. There was some recovery of total ATP production. These effects were most pronounced in the 1-5 μM range.

In glucose-treated cells but not in galactose-treated cells, treatment with talmapimod for 48 hours resulted in a dose-dependent increase in maximal respiration (11% increase at the 10u M concentration). However, unlike with pexmetinib treatment, there was no change in ATP production rates.

Unless otherwise defined, all technical and scientific terms used herein have the same meaning as commonly understood by one of ordinary skill in the art to which this invention belongs.

The inventions illustratively described herein may suitably be practiced in the absence of any element or elements, limitation or limitations, not specifically disclosed herein. Thus, for example, the terms “comprising”, “including,” “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed.

All publications, patent applications, patents, and other references mentioned herein are expressly incorporated by reference in their entirety, to the same extent as if each were incorporated by reference individually. In case of conflict, the present specification, including definitions, will control.

It is to be understood that while the disclosure has been described in conjunction with the above embodiments, that the foregoing description and examples are intended to illustrate and not limit the scope of the disclosure. Other aspects, advantages and modifications within the scope of the disclosure will be apparent to those skilled in the art to which the disclosure pertains.

What is claimed is:

1. A method for treating a disease or condition mediated, at least in part, by ECHS1 enzyme in a patient in need thereof, the method comprising administering a therapeutically effective amount of a compound selected from a p38 MAPK inhibitor, casein kinase inhibitor, TGF-β receptor inhibitor, RAF inhibitor, serine/threonine kinase inhibitor, TIE tyrosine kinase inhibitor, monocarboxylate transporter inhibitor, tyrosine phosphatase inhibitor, IAP inhibitor, CDK inhibitor, cytokine production inhibitor, Abl kinase inhibitor, bone morphogenetic protein inhibitor, AMPK inhibitor, MTH1 inhibitor, DYRK inhibitor, deubiquitinase inhibitor, ICAMI expression inhibitor, calcium channel blocker, glucosylglucose, trace amine-associated receptor antagonist, G protein-coupled receptor agonist, or a pharmaceutically acceptable salt thereof, or a combination thereof.

2. The method of claim 1, wherein the compound is ralimetinib, talmapimod, SB-239063, neflamapimod, SB-202190, TA-01, SB-242235, AL-8697, losmapimod, RepSox, tovorafenib, FRAX486, pexmetinib, adezmapimod, AZD3965, GW-1100, PPT, RWJ-67657, SX-011, felodipine, D-(+)-maltose, SKF-86002, TAK-715, bis(maltolato)oxovanadium(IV), EO-1428, SM-164, BMS-265246, semapimod, LY2109761, GDC-0879, PPY-A, K02288, WZ4003, SCH-51344, ID-8, AZD5582, CUDC-427, spautin-1, A205804, or SB590885, or a pharmaceutically acceptable salt thereof, or a combination thereof.

3. The method of claim 1, wherein the compound is a p38 MAPK inhibitor.

4. The method of claim 3, wherein the p38 MAPK inhibitor is selected from losmapimod, talmapimod, ralimetinib, pexmetinib, talmapimod, and neflamapimod.

5. The method of claim 1, wherein the disease or condition is a neurodegenerative disease or condition.

6. The method of claim 5, wherein the neurodegenerative disease or condition is Leigh syndrome.

7. The method of claim 1, wherein the patient is further administered a therapeutically effective amount of an additional therapeutic agent.

8. The method of claim 7, wherein the additional therapeutic agent is thiamine.

9. The method of claim 1, wherein the compound is administered in a pharmaceutical composition comprising a therapeutically effective amount of the compound and a pharmaceutically acceptable excipient.

10. The method of claim 1, wherein the patient is less than 3 years old.

11. A method for treating Leigh syndrome, the method comprising administering to a patient in need thereof, a therapeutically effective amount of a p38 MAPK inhibitor, or a pharmaceutically acceptable salt thereof.

12. The method of claim 11, wherein the p38 MAPK inhibitor is selected from losmapimod, talmapimod, ralimetinib, pexmetinib, talmapimod, or neflamapimod.

13. The method of claim 11, wherein the compound is administered in a pharmaceutical composition comprising a therapeutically effective amount of the compound and a pharmaceutically acceptable excipient.

14. The method of claim 11, wherein the patient is further administered a therapeutically effective amount of an additional therapeutic agent.

15. The method of claim 14, wherein the additional therapeutic agent is thiamine.

16. The method of claim 11, wherein the patient is less than 3 years old.

17. A method of treating Leigh syndrome comprising administering to a patient in need thereof a therapeutically effective amount of a compound which is selected from: 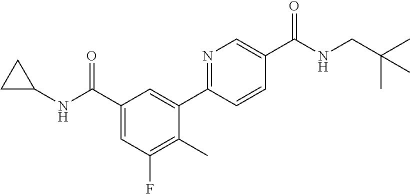 [SMILES: Cc1c(F)cc(C(=O)NC2CC2)cc1-c1ccc(C(=O)NCC(C)(C)C)cn1]
or a pharmaceutically acceptable salt thereof; 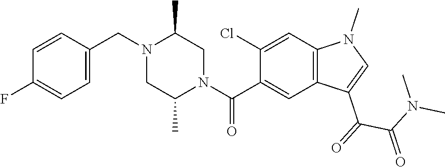 [SMILES: C[C@@H]1CN(Cc2ccc(F)cc2)[C@@H](C)CN1C(=O)c1cc2c(C(=O)C(=O)N(C)C)cn(C)c2cc1Cl]
or a pharmaceutically acceptable salt thereof; 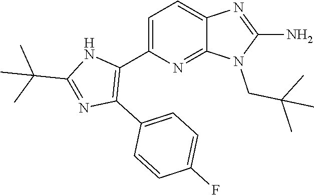 [SMILES: CC(C)(C)Cn1c(N)nc2ccc(-c3[nH]c(C(C)(C)C)nc3-c3ccc(F)cc3)nc21]
or a pharmaceutically acceptable salt thereof; 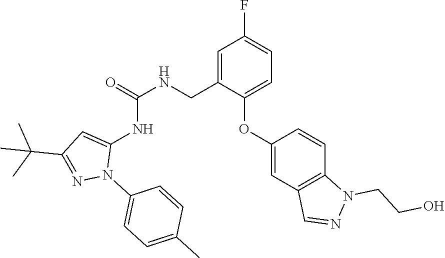 [SMILES: Cc1ccc(-n2nc(C(C)(C)C)cc2NC(=O)NCc2cc(F)ccc2Oc2ccc3c(cnn3CCO)c2)cc1]
or a pharmaceutically acceptable salt thereof; and 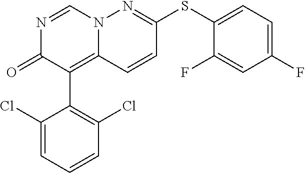 [SMILES: O=c1ncn2nc(Sc3ccc(F)cc3F)ccc2c1-c1c(Cl)cccc1Cl]
or a pharmaceutically acceptable salt thereof.

18. The method of claim 17, wherein the patient is further administered a therapeutically effective amount of an additional therapeutic agent.

19. The method of claim 18. wherein the additional therapeutic agent is thiamine.

20. The method of claim 17. wherein the patient is less than 3 years old.
